(19) United States
(12) Patent Application Publication
(10) Pub. No.: US 2004/0072865 A1
(43) Pub. Date: Apr. 15, 2004

(54) Aryl piperidine derivatives as inducers of ldl-receptor expression

(76) Inventors: Anne Marie Jeanne Bouillot, Les Ulis (FR); Agnes Bombrun, Geneva (CH); Bernard Andre Dumaitre, Les Ulis (FR); Rontain Luc Gosmini, Les Ulis (FR); Miles Stuart Congreve, Cambridge (GB); Nigel Grahame Ramsden, Hertfordshire (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/250,711

(22) PCT Filed: Jan. 15, 2001
(86) PCT No.: PCT/GB01/00155

(51) Int. Cl. A61K 31/4545; A61K 31/454; C07D 211/06; C 07D 4 1/02

(52) U.S. Cl. 514/317; 514/319; 514/326; 546/205; 546/207; 546/229

(57) ABSTRACT

The invention concerns a compound of formula (I), wherein Ar₁ represents phenyl, naphthyl or phenyl fused by a C₃₋₈cycloalkyl, where each group is substituted by a group —O-Z and optionally one to three further groups independently represented by R¹; Ar₂ represents phenyl or 5-6 membered heteroaromatic group, where each group is optionally substituted by one to four groups independently selected from halogen, C₁₋₄ alkyl and C₁₋₄ alkoxy; Ar₃ represents a phenyl or a 5-6 membered heteroaromatic group, where each group is optionally substituted by one to four groups independently selected from hydroxy, alkyl, C₁₋₄ alkoxy, C₂₋₄ alkenyl, C₂₋₄ alkenyloxy, C₁₋₄ perfluoroalkoxy, C₁₋₄ acylamino or an electron withdrawing group; A represents —C(H)—; E represents —C₁₋₆alkylene-; X represents —CON(H or C₁₋₄alkyl)- or —N(H or C₁₋₄alkyl)CO—; Y represents a direct link, —N(H or C₁₋₄alkyl)CO— or —CON(H or C₁₋₄alkyl)-; Z represents a metabolically labile group; R¹ represents halogen, —S(C₁₋₄alkyl), —O—(C₀₋₄ alkylene)-R² or —(C₀₋₄alkylene)-R², where each alkylene group may additionally incorporate an oxygen in the chain, with the proviso that there are at least two carbon atoms between any chain heteroatoms; R² represents: i) hydrogen, C₁₋₄ perfluoroalkyl, C₂₋₃alkenyl, ii) phenyl, naphthyl, a 5- or 6-membered heteroaromatic group or 1,2,3,4-tetrahydronaphthyl, optionally substituted by one or two halogen, hydroxy, C₁₋₄ alkyl, C₁₋₄ alkoxy groups, iii) C₃₋₈cycloalkyl, a 3-7 membered heterocycloalkyl, iv) amino, C₁₋₄ alkylamino or di-C₁₋₄alkylamino, with the proviso that there are at least two carbon atoms between any chain heteroatoms; and physiologically acceptable salts, solvates and pharmaceutical compositions thereof and their use in treating disorders associated with elevated circulating levels of LDL-cholesterol.

This invention relates to novel compounds which up-regulate LDL receptor (LDL-r) expression and to processes for their preparation, pharmaceutical compositions containing them and their medical use. More particularly, this invention relates to novel aromatic piperidines and their use in therapy.

Epidemiological studies have clearly demonstrated the correlation between reduction in plasmatic LDL cholesterol and the benefit on cardiovascular events including mortality. LDL cholesterol is eliminated from plasma by specific binding to LDL-r expressed by the liver. Regulation of LDL-r expression occurs in the liver and is mainly dependent on intracellular cholesterol concentration. Increasing free cholesterol concentration leads to a reduced LDL-r expression through a mechanism involving transcriptional factors. Counteracting with this process is expected to up-regulate LDL-r expression in the liver and to increase the clearance of LDL cholesterol.

International Patent Application Number PCT.EP00.06668 concerns the novel use of the SREBP-cleavage activating protein (SCAP) in a screening method, and two compounds are disclosed, namely 4-(4-chloro-benzoylamino)-N{4-[4-(2-ethoxy-4-ethyl-phenyl)-piperidin-1-yl]-butyl}-benzamide and 4-(4-Benzoyl)-N-{4-[4-(4-isopropyl-2-methoxy-phenyl)piperidin-1-yl]-butyl}-benzamide hydrochloride, which do not form part of the present invention.

Another publication, Bioorganic and Medicinal Chemistry Letters Vol. 5, 3, 219-222, 1995 discloses compounds having the general formula (A)

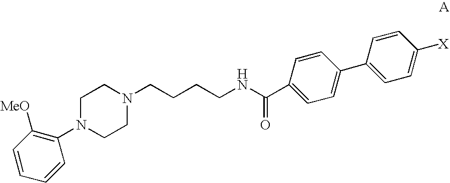

where X may be COMe, SO₂Me and NH₂, as having high affinity for the dopamine D₃ receptor and postulates their use in CNS disorders, particularly psychiatric illness. The compound of formula A where X is COMe is also disclosed in J. Pharmacol. Exp. Ther. 287; 1 1998 187-197 and Bioorganic and Medicinal Chemistry Letters Vol. 7, 15, 1995-1998, 1997, again as being useful in treating CNS disorders. It will be noted that the examples of the present invention differ from those of formula (A) in use of a piperidine ring rather than a piperazine and in the utility disclosed.

Journal Of Medicinal Chemistry, Vol. 40,6,952-960, 1997 discloses compounds of formula (B)

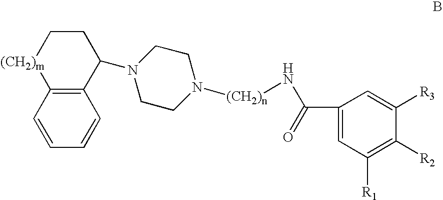

where m=0, 1 or 2; n=2 or 3; R₁ and R₃=H or OMe and R² may be Ph, as selective 5-HT_{1A} receptor ligands having CNS activity. It will be noted that the examples of the present invention differ from those of formula (B) in use of a piperidine ring rather than a piperazine and in the utility disclosed.

International Patent Application Publication Number WO99/45925 discloses compounds of formula (C)

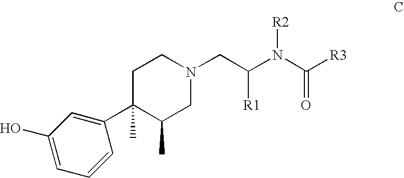

where R1 may be hydrogen, R2 may be hydrogen and R3 may be a group

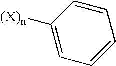

where X may be an aryl group and n may be 1. Specifically disclosed are compounds where the group COR3 is formed from 2- and 4-biphenyl carboxylic acid and R1 and R2 are methyl or hydrogen respectively. The utility of the compounds is as opioid receptor binding agents which may be useful as analgesics. The substitution on the 3- and 4-positions of the piperidine ring leave the compounds of this publication outside the scope of the present invention. Furhtermore, the utility disclosed is different.

International Patent Application Publication Number WO98/37893 discloses compounds of formula (D)

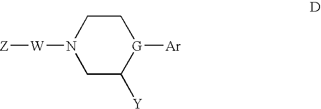

where Ar may represent an optionally substituted phenyl or naphthyl, G may be N or CH₂ (sic), W may be an optionally substituted alkylene, Y may be hydrogen and Z may represent a group R₄CONR₅, where R₄ may be an optionally substituted phenyl and R₅ may be hydrogen. These compounds are described as being D2 receptor antagonists useful in the treatment of CNS disorders such as Parkinson's Disease. None of the compounds specifically disclosed fall within the scope of the present invention and the disclosed utlity is different.

International Patent Application Publication Number WO9402473 discloses compounds of formula (E)

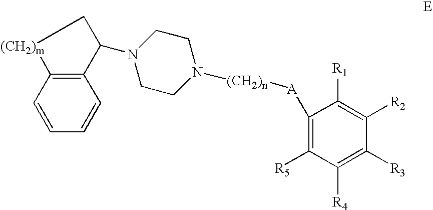

where A may be NHCO or CONH; R₁-R₅ may be hydrogen or a benzene ring, m may be 1-3 and n may be 1-3. Specifically disclosed are compounds

| No. | A | n | m | R₁ | R₂ | R₃ | R₄ | R₅ |
|---|---|---|---|---|---|---|---|---|
| 5 | NHCO | 2 | 1 | H | H | Ph | H | H |
| 12 | NHCO | 2 | 2 | H | H | Ph | H | H |
| 19 | NHCO | 2 | 3 | H | H | Ph | H | H |

The compounds are described as 5HT-1A agonists having CNS activity and may be used as anti-depressants, anti-hypertensive, analgesics etc. It will be noted that the examples of the present invention differ from those of formula (E) in use of a piperidine ring rather than a piperazine and in the utility disclosed.

International Patent Application Publication Number WO99/45925 discloses compounds of formula (F)

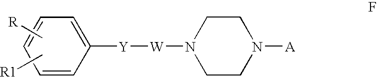

where A may represent a substituted phenyl group, W represents a linear or branched alkylene group having from 2 to 6 carbon atoms; Y may represent a group NHCO or CONH; and R may be a substituted phenyl group. Particularly disclosed is the compound G

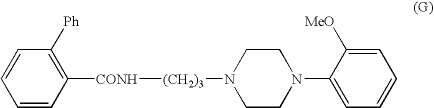

These compounds are described as being α1A-adrenergic receptors useful in the treatment of contractions of the prostate, urethra and lower urinary tract, without affecting blood pressure. It will be noted that the examples of the present invention differ from those of formula (G) in use of a piperidine ring rather than a piperazine and in the utility disclosed.

International Patent Application Publication Number WO98/35957 describes compounds of formula (H)

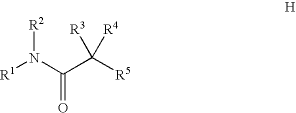

wherein R1-R5 are each individually selected from the group of substituents including hydrogen, halogen, hydroxyl, thiol, lower alkyl, substituted lower alkyl, alkenyl, alkynyl, alkylalkenyl, alkylalkynyl, alkoxy, alkylthio, acyl, aryloxy, amino, amido, carboxyl, aryl, substituted aryl, heterocycle, heteroaryl, substituted heterocycle, heteroalkyl, cycloalkyl, substituted cycloalkyl, alkylcycloalkyl, alkylcycloheteroalkyl, nitro and cyano. Specifically disclosed compounds are those formed by the N-alkylation of a a substituted piperidine or piperazine with a group (J)

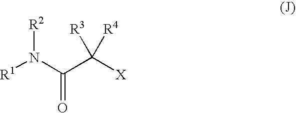

where X is a leaving group. None of the compounds specifically disclosed fall within the scope of the present invention and the invention is in no way suggested by the disclosure. The compounds are said to be of use as NPY Y5 receptor antagonists in the treatment of obesity, bulemia and related disorders and NPY Y5 receptor inhibition related disorders such as memory disorders, epilepsy, dyslipidemia and depression. U.S. Pat. No. 6,048,900, published after the priority date of the present invention discloses the same information.

Journal Of Medicinal Chemistry, Vol. 31, 1968-1971, 1988 discloses certain aryl piperazines compounds, which fall outside the present invention, as 5HT-1a Serotonin Ligands as potential CNS agents. Specifically disclosed are compounds of formula (K)

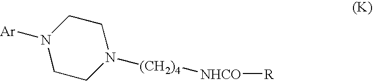

where Ar=Ph and R=Ph, Ar=2—OMePh and R=Ph and Ar=2-pyrimidyl and R=Ph.

Journal Of Medicinal Chemistry, Vol. 34, 2633-2638, 1991 discloses aryl piperazines having reduced al adrenergic affinity. Specifically disclosed is the compound (L)

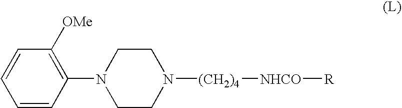

where R is 4—(BnO)-phenyl, which falls outside the scope of the present invention.

UK Patent Application Number 0003192.2, which provides background to the disclosure of the present invention, discloses compounds of formula (X)

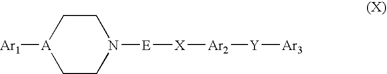

wherein

Ar₁ represents a phenyl, naphthyl or phenyl fused by a C₃₋₈cycloalkyl, where each group is optionally substituted by methylenedioxy or one to four groups independently represented by R¹;

Ar₂ represents a phenyl or 5-6 membered heteroaromatic group, optionally substituted by one to four groups independently selected from halogen, C₁₋₄ alkyl and C₁₋₄ alkoxy;

Ar₃ represents a phenyl or a 5-6 membered heteroaromatic group, optionally substituted by one to four groups independently selected from halogen, hydroxy, nitrile C₁₋₄ alkyl, C₁₋₄ alkoxy, C₂₋₄ alkenyl, C₂₋₄ alkenyloxy, C₁₋₄ perfluoroalkyl, C₁₋₄ perfluoroalkoxy, C₁₋₄ acyl, C₁₋₄ alkoxycarbonyl, aminocarbonyl, C₁₋₄ alkylaminocarbonyl; di-C₁₋₄ alkylaminocarbonyl and C₁₋₄ acylamino;

A represents —C(H or C₁₋₄ alkyl)- or —N—;

E represents —C₁₋₆ alkylene-;

X represents —CON(H or C₁₋₄ alkyl)- or —N(H or C₁₋₄alkyl)CO—;

Y represents a direct link, —N(H or C₁₋₄alkyl)CO— or —CON(H or C₁₋₄alkyl)-;

R¹ represents halogen, —O—(C₀₋₄ alkylene)-R² or —(C₀₋₄alkylene)-R², where each alkylene group may additionally incorporate an oxygen in the chain, with the proviso that there are at least two carbon atoms between any chain heteroatoms;

R² represents

(i) hydrogen, C₁₋₄ perfluoroalkyl,

(ii) phenyl, naphthyl, a 5- or 6-membered heteroaromatic group or 1,2,3,4-tetrahydronaphthyl, optionally substituted by one or two halogen, hydroxy, C₁₋₄ alkyl, C₁₋₄ alkoxy groups,

(iii) C₃₋₈cycloalkyl, a 3-7 membered heterocycloalkyl,

(iv) amino, C₁₋₄ alkylamino or di-C₁₋₄alkylamino, with the proviso that there are at least two carbon atoms between any chain heteroatoms;

or a physiologically acceptable salt, solvate or derivative thereof. Specifically disclosed are the following compounds:

4-(4-chloro-benzoylamino)-N{4-[4-(5-methyl-2-piperidin-4-yl-phenol)]-butyl}-benzamide;

4-(4-chloro-benzoylamino)-N{4-[4-(2-ethoxy-4-methyl-phenyl)-piperidin-1-yl]-butyl}-benzamide;

4′-Trifluoromethyl-biphenyl-4-carboxylic acid {4-[4-(2-ethoxy-4-methyl-phenyl)-piperidin-1-yl]-butyl}amide;

4′-Trifluoromethyl-biphenyl-4-carboxylic acid (4-{4-[2,5-dimethyl-4-(pyridin-2-ylmethoxy)-phenyl]-piperidin-1-yl}butyl)-amide;

4-Methyl-2-(4-trifluoromethyl-phenyl)-thiazole-5-carboxylic acid {4-[4-(4-isopropyl-2-methoxy-phenyl)-piperidin-1-yl]-butyl}-amide;

4-(4-chloro-benzoylamino)-N-{4-[4-(2,4-dimethoxy-phenyl)-piperidin-1-yl]-butyl}-benzamide;

4-(4-chloro-benzoylamino)-N{4-[4-(2,4-diethoxy-phenyl)-piperidin-1-yl]-butyl}-benzamide;

4-(4-chloro-benzoylamino)-N-[4-(4-benzo[1,3]dioxol-5-yl-piperidin-1-yl]-butyl)}-benzamide;

4-(4-chloro-benzoylamino)-N-[4-(4-naphthalen-1-yl-piperidin-1-yl]-butyl}benzamide;

4-(4-chloro-benzoylamino)-N-{4-[4-(5,6,7,8-tetrahydro-naphthalen-2-yl)-piperidin-1-yl]-butyl}benzamide;

4′-Trifluoromethyl-biphenyl-4-carboxylic acid [4-[4-(2-cyclopropylmethoxy-4-ethyl-phenyl)-piperidin-1-yl]-butyl]-amide;

4′-Chloro-biphenyl-4-carboxylic acid {4-[4-(1-methoxy-naphtalen-2-yl)-piperidin-1-yl]-butyl}-amide;

4-(4-chloro-benzoylamino)-N-{4-[4-(2-trifluoroethoxy-4-methyl-phenyl)-piperidin-1-yl]-butyl}-benzamide;

4-(4-chloro-benzoylamino)-N{4-[4-(2-methoxy-4-methyl-phenyl)-piperazin-1-yl]-butyl}-benzamide;

or a physiologically acceptable salt, solvate or derivative thereof, all of which are indicative of the range of substitution which may be tolerated on compounds according to the present invention as additionally exemplified by the non-limiting examples disclosed herein.

The present invention concerns pro-drugs of certain compounds related to UK Patent Application Number 0003192.2 and their utility in the field of medicine.

Thus, the present invention provides a compound of formula (I)

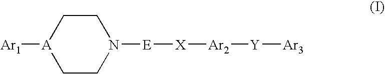

wherein

Ar₁ represents phenyl, naphthyl or phenyl fused by a C₃₋₈cycloalkyl, where each group is substituted by a group —O-Z and optionally one to three further groups independently represented by R¹;

Ar₂ represents phenyl or 5-6 membered heteroaromatic group, where each group is optionally substituted by one to four groups independently selected from halogen, C₁₋₄ alkyl and C₁₋₄ alkoxy;

Ar₃ represents a phenyl or a 5-6 membered heteroaromatic group, where each group is optionally substituted by one to four groups independently selected from hydroxy, alkyl, C₁₋₄ alkoxy, C₂₋₄ alkenyl, C₂₋₄ alkenyloxy, C₁₋₄ perfluoroalkoxy, C₁₋₄ acylamino or an electron withdrawing group;

A represents —C(H)—;

E represents —C₁₋₆ alkylene-;

X represents —CON(H or C₁₋₄alkyl)- or —N(H or C₁₋₄alkyl)CO—;

Y represents a direct link, —N(H or C₁₋₄alkyl)CO— or —CON(H or C₁₋₄alkyl)-;

Z represents a metabolically labile group;

R¹ represents halogen, —S(C₁₋₄alkyl), —O—(C₀₋₄ alkylene)-R² or —C₀₋₄alkylene)-R², where each alkylene group may additionally incorporate an oxygen in the chain, with the proviso that there are at least two carbon atoms between any chain heteroatoms;

R² represents

(i) hydrogen, C₁₋₄ perfluoroalkyl, C₂₋₃alkenyl,

(ii) phenyl, naphthyl, a 5- or 6-membered heteroaromatic group or 1,2,3,4-tetrahydronaphthyl, optionally substituted by one or two halogen, hydroxy, C₁₋₄ alkyl, C₁₋₄ alkoxy groups,

(iii) C₃₋₈cycloalkyl, a 3-7 membered heterocycloalkyl,

(iv) amino, C₁₋₄ alkylamino or di-C₁₋₄alkylamino, with the proviso that there are at least two carbon atoms between any chain heteroatoms;

or a physiologically acceptable salt or solvate thereof.

Suitable physiologically acceptable salts of the compounds of general formula (I) include acid addition salts formed with pharmaceutically acceptable inorganic acids for example, hydrochlorides, hydrobromides or sulphates, or with pharmaceutically acceptable organic acids for example mesylates, lactates and acetates. More suitably, a physiologically acceptable salt of the compounds of general formula (I) is a mesylate salt.

The solvates may, for example, be hydrates.

References herein after to a compound according to the invention include both compounds of formula (I) and their physiologically acceptable salts together with physiologically acceptable solvates.

Referring to the general formula (I), alkyl, alkylene and alkoxy include both straight and branched chain saturated hydrocarbon groups. Examples of alkyl groups include methyl and ethyl groups, examples of alkylene groups include methylene and ethylene groups, whilst examples of alkoxy groups include methoxy and ethoxy groups.

Referring to the general formula (I), alkenyl includes both straight and branched chain saturated hydrocarbon groups containing one double bond. Examples of alkenyl groups include ethenyl or n-propenyl groups.

Referring to the general formula (I), acyl refers to aliphatic or cyclic hydrocarbons attached to a carbonyl group through which the substituent bonds, such as acetyl.

Referring to the general formula (I), phenyl fused by a C₃₋₈cycloalkyl includes bicyclic rings such as 1,2,3,4-tetrahydronaphthyl, which, for the avoidance of doubt, is linked to the rest of the molecule through the aromatic ring.

Referring to general formula (I), a halogen atom includes fluorine, chlorine, bromine or iodine.

Referring to the general formula (I), C₁₋₃perfluoroalkyl and C₁₋₃perfluoroalkoxy includes compounds which the hydrogens have been partially or fully replaced by fluorines, such as trifluoromethyl and trifluoromethoxy or trifluoroethyl.

Referring to the general formula (I), a 5-6 membered hetroaromatic group includes a single aromatic ring system containing at least one ring heteroatom independently selected from O, N and S. Suitable examples include pyridyl and thiazolyl.

Referring to the general formula (I), reference to a C₃₋₈ cycloalkyl group means any single carbocyclic ring system, wherein said ring is fully or partially saturated. Suitable examples include cyclopropyl and cyclohexyl groups.

Referring to the general formula (I), reference to a 3-7 membered heterocycloalkyl group means any single ring system containing at least one ring heteroatom independently selected from O, N and S, wherein said ring is fully or partially saturated.

Suitably, Ar₁ represents a phenyl, naphthyl or 1,2,3,4-tetrahydronaphthyl group, substituted by a group —O-Z, where further optional substititution is effected by R¹. More suitably, Ar₁ represents a substituted phenyl or naphthyl. Preferably Ar₁ represents a substituted phenyl. Equally preferably, Ar₁ represents a substituted naphthyl.

Substitution on Ar₁ is suitably represented by —O-Z and one, two or three further groups independently selected from C₁₋₄ alkyl, e.g. methyl, ethyl or iso-propyl, C₁₋₄ alkoxy; e.g. methoxy or ethoxy, —O—Cₒ₋₄alkene-R², e.g. —O-methylene-R², where R² represents C₁₋₄ perfluoroalkyl, e.g. trifluoromethyl, a 5-6 membered heteroaromatic group, e.g. pyridyl, preferably 2-pyridyl, or a C₃₋₈cycloalkyl, e.g. cyclopropyl.

Substitution on Ar₁ is equally suitably represented by one or two groups independently selected from C₁₋₄ alkyl, e.g. methyl or ethyl, C₁₋₄ alkoxy, e.g. methoxy, ethoxy, propoxy, isobutoxy, C₂₋₃alkenyloxy, e.g. allyloxy, —O-C₀₋₄alkylene-R², e.g. —O-methylene-R², where R² represents a C₃₋₈cycloalkyl, e.g. cyclopropyl.

Preferably, Ar₁ is a phenyl group substituted by —O-Z and optionally one or two further groups independently selected from methyl, ethyl, isopropyl, methoxy, ethoxy, cyclopropylmethoxy and 2-pyridylmethoxy. Preferably, substitution is in two or three of the 2-, 4- or 5-positions on the phenyl ring.

Equally preferably, Ar₁ is a phenyl or naphthyl group substituted by —O-Z and optionally one further group selected from methyl, ethyl, methoxy, ethoxy, propoxy, isobutoxy, allyloxy and cyclopropylmethoxy. Preferably, where Ar₁ is phenyl, substitution is in one or two of the 2- or 4-positions on the phenyl ring. Preferably, where Ar₁ is naphthyl, the link to group A is through the 1- or 2-position and mono-substitution by R¹ is in either the corresponding 1- or 2-positions.

E is preferably an n-butylene group.

X is suitably a —N(H or C₁₋₄ alkyl)CO— group, preferably an —N(H)CO— group.

Y is suitably an —N(H or C₁₋₄ alkyl)CO— group or a direct link. Preferably, Y is an —N(H or C₁₋₄ alkyl)CO— group. Equally preferably, Y is a direct link.

The group Z is any metabolically labile group which may be cleaved upon administration to a mammal, such as a human, to give the corresponding —OH derivative. For the avoidance of any doubt, compounds of formula (I) have activity in their own right. Such Z groups are clear to those skilled in the art, without undue experimentation, and with reference to the teaching of Burger's Medicinal Chemistry And Drug Discovery, 5th Edition, Vol 1: Principles And Practice, the principle details relating to prodrugs and metabolites of which is incorporated herein by reference. Suitable Z groups include C₁₋₄ acyl, C₁₋₄ acyloxymethylene, optionally substituted benzoyl, where optional substitution may be effected by one or more C₁₋₄ alkyl, halogen, hydroxy or C₁₋₄ alkoxy, —PO(OR³)₂, where R³ represents hydrogen, C₁₋₄ alkyl, phenyl or phenylmethyl, carboxyethylcarbonyl, C₁₋₄ alkylaminocarbonyl, C₁₋₄ dialkylaminocarbonyl or esters formed with readily available amino acids, e.g. dimethylaminomethylcarbonyl. Z is more sutiably C₁₋₄ acyl, e.g. acetyl or —PO(OR³)₂, where R³ represents hydrogen, C₁₋₄ alkyl, phenyl or phenylmethyl, e.g. phosphate.

The group —O-Z is suitably ortho-substituted to the link to the group A.

Where Ar₂ is a 5-6-membered heteroaromatic group, this is suitably a thiazolyl group, optionally substituted by C₁₋₄ alkyl, e.g. methyl. Ar₂ is preferably para substituted phenyl.

Suitable electron withdrawing groups on Ar₃ include halogen, nitrile, nitro, C₁₋₄, C₁₋₄ perfluoroalkyl, C₁₋₄ acyl, C₁₋₄ alkoxycarbonyl, aminocarbonyl, C₁₋₄ alkylaminocarbonyl; di-C₁₋₄ alkylaminocarbonyl, C₁₋₄ alkylsulfonyl, C₁₋₄ alkylaminosulfonyl and di-C₁₋₄ alkylaminosulfonyl, C₁₋₄ alkylsulfonyl and C₁₋₄alkylsulfoxy.

Ar₃ is preferably phenyl or a pyridyl group, suitably 2-pyridyl, substituted by a halogen, e.g. chloro or C₁₋₄perfluoroalkyl, e.g. trifluoromethyl.

Ar₃ is equally preferably phenyl substituted by a halogen, e.g. chloro, C₁₋₄perfluoroalkyl, e.g. trifluoromethyl, nitrile or C₁₋₄alkylsulfonyl, e.g. methylsulfonyl.

When Ar₃ is phenyl, para-substitution is preferred.

More preferably, Ar₃ is phenyl substituted by a halogen, e.g. chloro or nitrile. Most preferably, Ar₃ is phenyl substituted by a halogen, e.g. chloro.

Particularly preferred compounds of the invention include those in which each variable in Formula (I) is selected from the preferred groups for each variable. Even more preferable compounds of the invention include those where each variable in Formula (I) is selected from the more preferred or most preferred groups for each variable.

A preferred sub-group of a compound of formula (I) is represented by a compound of formula (Ia)

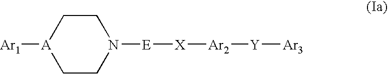

wherein

Ar₁ represents phenyl, naphthyl or phenyl fused by a C₃₋₈cycloalkyl, where each group is substituted by a group —O-Z and optionally one to three further groups independently represented by R¹;

Ar₂ represents a phenyl or 5-6 membered heteroaromatic group, where each group is optionally substituted by one to four groups independently selected from halogen, C₁₋₄ alkyl and C₁₋₄ alkoxy;

Ar₃ represents a phenyl or a 5-6 membered heteroaromatic group, where each group is optionally substituted by one to four groups independently selected from halogen, hydroxy, nitrile, C₁₋₄ alkyl, C₁₋₄alkoxy, C₂₋₄ alkenyl, C₂₋₄ alkenyloxy, C₁₋₄ perfluoroalkyl, C₁₋₄ perfluoralkoxy, C₁₋₄ acyl, C₁₋₄ alkoxycarbonyl, aminocarbonyl, C₁₋₄ alkylaminocarbonyl; di-C₁₋₄ alkylaminocarbonyl and C₁₋₄ acylamino;

A represents —C(H)—;

E represents —C₁₋₆ alkylene-;

X represents —CON(H or C₁₋₄alkyl)- or —N(H or C₁₋₄alkyl)CO—;

Y represents a direct link, —N(H or C₁alkyl)CO— or —CON(H or C₁₋₄alkyl)-;

Z represents a metabolically labile group;

R¹ represents halogen, —O—(C₀₋₄ alkylene)-R² or (C₀₋₄alkylene)-R², where each alkylene group may additionally incorporate an oxygen in the chain, with the proviso that there are at least two carbon atoms between any chain heteroatoms;

R² represents

(i) hydrogen, C₁₋₄ perfluoroalkyl,

(ii) phenyl, naphthyl, a 5- or 6-membered heteroaromatic group or 1,2,3,4-tetrahydronaphthyl, optionally substituted by one or two halogen, hydroxy, C₁₋₄ alkyl, C₁₋₄ alkoxy groups,

(iii) C₃₋₈cycloalkyl, a 3-7 membered heterocycloalkyl,

(iv) amino, C₁₋₄ alkylamino or di-C₁₋₄alkylamino, with the proviso there are at least two carbon atoms between any chain heteroatoms;

or a physiologically acceptable salt or solvate thereof.

A further preferred sub-group of the present invention is represented by a compound of formula (Ib)

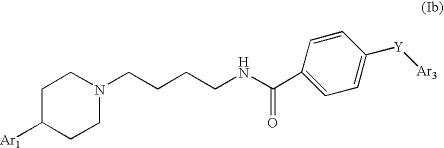

wherein

Ar₁ represents phenyl, naphthyl or 1,2,3,4-tetrahydronaphthyl, where each group is substituted in by a group —O-Z and optionally by one or two further groups independently represented by R¹;

Ar₃ represents phenyl substituted in the para position by a halogen, nitrile or C₁₋₄ perfluoroalkyl group;

R¹ represents halogen, C₁₋₄ alkyl, C₁₋₄ alkyl or —O—(C₀₋₄alkylene)-R²,

R² represents hydrogen, C₁₋₄ perfluoroalkyl, a 5- or 6-membered heteroaromatic group or C₃₋₈cycloalkyl;

Y represents a direct link or —N(H)CO—;

Z represents a C₁₋₄acyl group or a PO(OR³)₂, where R³ represents hydrogen, C₁₋₄ alkyl, phenyl or phenylmethyl;

or a physiologically acceptable salt or solvate thereof.

It will be understood that references to compounds of formula (I) hereinbefore and hereinafter apply equally to compounds of formula (Ia) and (Ib).

Suitable compounds according to the invention include:

Acetic acid 2-(1-{4-[4-(4-chloro-benzoylamino)-benzoylamino]-butyl}piperidin-4-yl)-5-methyl-phenyl ester;

Phosphoric acid 2-(1-{4-[4-(4-chloro-benzoylamino)-benzoylamino]-butyl}-piperidin-4-yl)-5-methyl-phenyl ester diethyl ester;

Acetic acid 2-(1-{4-[4-(4-chloro-benzoylamino)-benzoylamino]-butyl}-piperidin-4-yl)-5-ethyl-phenyl ester;

Acetic acid 2-(1-{4-[(4′-cyano-biphenyl-4-carbonyl)-amino]-butyl}piperidin-4-yl)-5-ethyl-phenyl ester;

Acetic acid 2-(1 {4-[4-(4-chloro-benzoylamino)-benzoylamino]-butyl}-piperidin-4-yl)-5,6,7,8-tetrahydro-naphtalen-1-yl ester;

Acetic acid 2-(1-{4-[(4′-cyano-biphenyl-4-carbonyl)-amino]-butyl}piperidin-4-yl)-5,6,7,8-tetrahydro-naphtalen-1-yl ester;

Acetic acid 2-(1-{4-[4-(4-chloro-benzoylamino)-benzoylamino]-butyl}-piperidin-4-yl)-naphtalen-1-yl ester;

Acetic acid 2-(1-{4-[(4′-cyano-biphenyl-4-carbonyl)-amino]-butyl}-piperidin-4-yl)-naphtalen-1-yl ester,

Phosphoric acid 2-(1-{4-[(4′-cyano-biphenyl-4-carbonyl)-amino]-butyl}-piperidin-4-yl)-5-methyl-phenyl ester diethyl ester;

Phosphoric acid mono-[2-(1-{4-[(4′-cyano-biphenyl-4-carbonyl)-amino]-butyl}-piperidin-4-yl)-5-methyl-phenyl]ester;

Phosphoric acid 2-(1-{1-[(4′-cyano-biphenyl-4-carbonyl)-amino]-butyl}-piperidin-4-yl)-5,6,7,8-tetrahydro-naphtalen-1-yl ester diethyl ester;

Acetic acid 2-(1-{4-[(4′-cyano-biphenyl-4-carbonyl)-amino]-butyl}-piperidinyl)-5-methyl-phenyl ester;

Phosphoric acid 2-(1-{4-[4-(4-chloro-benzoylamino)-benzoylamino]-butyl}-piperidin-4-yl)-5,6,7,8-tetrahydro-naphtalen-1-yl ester diethyl ester;

Phosphoric acid 2-(1-{4-[4-(4-chloro-benzoylamino)-benzoylamino]-butyl}-piperidin-4-yl)-5-ethyl-phenyl ester diethyl ester;

or a physiologically acceptable salt or solvate thereof.

The compounds of the invention are inductors of LDL-r expression and are thus of use in the treatment of conditions resulting from elevated circulating levels of LDL-cholesterol.

The ability of the compounds of the invention to induce LDL-r expression by human hepatocytes in vitro is determined using a human hepatocarcinoma cell line, Hep G2, as a model system. A reporter gene assay using the LDL-r promotor in front of the reporter gene luciferase is used as a primary screen.

The in vivo profile of the compounds is evaluated by oral administration of the compounds of the invention to fat-fed hamsters. Measurements of VLDL/LDL cholesterol and triglycerides upon treatment allow to determine the activity.

The compounds of the invention are potent and specific inductors of LDL-r expression, which furthermore exhibit good oral bioavailability and duration of action.

Compounds of the invention are of use in the treatment of diseases in which lipid imbalance is important, e.g. atherosclerosis, pancreatitis, non-insulin dependent diabetes mellitus (NIDDM), coronary heart diseases and obesity.

Compounds of the invention are also useful in lowering serum lipid levels, cholesterol and/or triglycerides, and are of use in the treatment of hyperlipemia, hyperlipidemia, hyperlipoproteinemia, hypercholesterolemia and/or hypertriglyceridemia.

The invention therefore provides a compound of formula (I) or a physiologically acceptable salt or solvate thereof for use in therapy, in particular in human medicine.

There is also provided as a further aspect of the invention the use of a compound of formula (I) or a physiologically acceptable salt or solvate thereof in the preparation of a medicament for use in the treatment of conditions resulting from elevated circulating levels of LDL-cholesterol.

In an alternative or further aspect there is provided a method for the treatment of a mammal, including man, in particular in the treatment of conditions resulting from elevated circulating levels of LDL-cholesterol, comprising administration of an effective amount of a compound of formula (I) or a physiologically acceptable salt or solvate thereof.

It will be appreciated that reference to treatment is intended to include prophylaxis as well as the alleviation of established symptoms; Compounds of formula (I) maybe administered as the raw chemical but the active ingredient is preferably presented as a pharmaceutical formulation.

Accordingly, the invention also provides a pharmaceutical composition which comprises at least one compound of formula (I) or a physiologically acceptable salt or solvate thereof and formulated for administration by any convenient route. Such compositions are preferably in a form adapted for use in medicine, in particular human medicine, and can conveniently be formulated in a conventional manner using one or more pharmaceutically acceptable carriers or excipients.

Thus compounds of formula (I) may be formulated for oral, buccal, parenteral, transdermal, topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or lozenges formulated in conventional manner.

For transdermal administration the compounds according to the invention may be formulated as creams, gels, ointments or lotions or as a transdermal patch. Such compositions may for example be formulated with an aqueous or oily base with the addition of suitable thickening, gelling, emulsifying, stabilising, dispersing, suspending, and/or colouring agents.

The compounds of the invention may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unit dose device or alternatively as a powder mix with a suitable carrier for administration using a suitable delivery device.

The compositions may contain from 0.1% upwards, e.g. 0.1-99% of the active material, depending on the method of administration. A proposed dose of the compounds of the invention is 0.25 mg/kg to about 125 mg/kg bodyweight per day e.g. 20 mg/kg to 100 mg/kg per day. It will be appreciated that it may be necessary to make routine variations to the dosage, depending on the age and condition of the patient and the precise dosage will be ultimately at the discretion of the attendant physician or veterinarian. The dosage will also depend on the route of administration, and the particular compound selected.

The compounds of formula (I) may, if desired, be administered with one or more therapeutic agents and formulated for administration by any convenient route in a conventional manner. Appropriate doses will be readily appreciated by those skilled in the art. For example, the compounds of formula (I) may be administered in combination with an HMG CoA reductase inhibitor, an agent for inhibition of bile acid transport or fibrates.

A compound of formula (I), or a physiologically acceptable salt, solvate or derivative thereof, may be prepared by the general methods outlined hereafter. In the following description, the groups Ar₁, Ar₂, Ar₃, R¹, R², A, E and X are as previously defined for compounds of formula (I), unless specified otherwise.

According to a first general process (A), a compound of formula (I) may be prepared by reaction of a compound of formula (II) with a compound of formula (III)

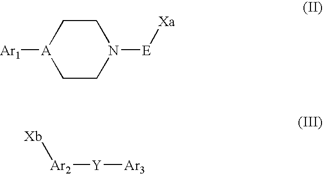

where Xa and Xb are suitable reactants to form a group X. For example, where X is N(H or C₁₋₄ alkyl)CO, Xa is NH₂ or NH(C₁₋₄ alkyl) and Xb is COL where L is OH or a suitable leaving group, such as halide. Such a reaction may be effected under standard amide bond-forming conditions, including those described herein.

A compound of formula (II) where Xa is NH₂ or NH(C₁₋₄ alkyl), may be prepared by reaction of a compound of formula (IV) with a compound of formula (V)

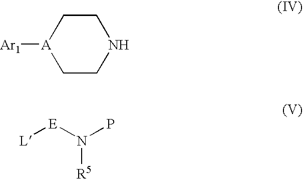

where R⁵ represents H or C₁₋₄alkyl, L′ is a suitable group, such as halide, and P is any suitable N-protecting group, under standard alkylation conditions, including those described herein, followed by removal of the protecting group under standard conditions.

A compound of formula (II) where Xa is NH₂ or NH(C₁₋₄ alkyl), may further be prepared by reaction of a compound of formula (IV) with a compound of formula (Va)

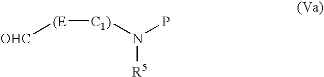

where R⁵ represents H or C₁₋₄alkyl, E-C₁ (‘E minus C₁’) represents the group E with one less carbon group in its chain and P is any suitable N-protecting group, under standard reductive amination conditions, including those described herein, followed by removal of the protecting group under standard conditions.

A compound of formula (IV), may be prepared by reaction of a compound Ar₁-sal, where sal represents the lithium or magnesium ion of Ar₁, with a compound of formula (VI)

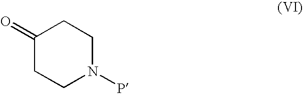

where P′ represents a suitable N-protecting group, such as acetyl, benzyl or benzyl-4-oxo-1 carboxylate, followed by the steps of dehydration, reduction of the resulting double bond, and finally, removal of the protecting group P′. Such chemistry has been described, for example, in European Patent Appliction no. 0630887.

Alternatively, a compound of formula (IV) where Ar₁ is substituted by an activated ortho or para activating group for the reaction centre, Act, e.g. methoxy or hydroxy, may be prepared by reaction of a compound of formula Ar₁-Act, with a compound of formula (VI) under suitable reaction conditions such as e.g. trifluoroborane or acetic acid and aqueous hydrochloric acid, to form a tetrahydropyridyl ring, followed by reduction, e.g. under hydrogenation conditions, of the resulting double bond and finally deprotection of the N-protecting group, P′ under standard conditions.

Alternatively, a compound of formula (I) where where Ar₁ is substituted by an activated ortho or para activating group for the reaction centre, Act, e.g. methoxy or hydroxy may be prepared by reaction of a compound of formula Ar₁-Act, with a compound of formula (VII)

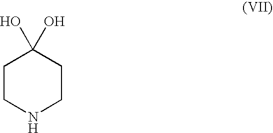

under suitable reaction conditions such as e.g. acetic acid and aqueous hydrochloric acid to form a tetrahydropyridyl ring, followed by suitable N-protection, then reduction, e.g. under hydrogenation conditions, of the resulting double bond and finally deprotection of the N-protecting group.

A compound of formula (III) may be prepared by standard methods including, where Xb is CO₂H, deprotection of a compound of formula (X)

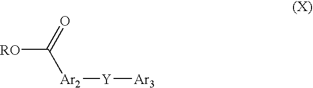

where R is a suitable carboxylic acid protecting group, such as methyl.

A compound of formula (X) where R is OH or a suitable protecting group and Y is a direct link, may be prepared by reaction of a compound of formula (XI), with a compound of formula (XII)

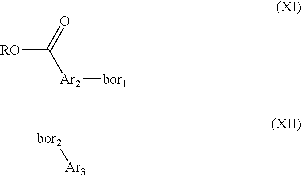

where bor₁ represents a boronic acid group or a halide, e.g. bromide or iodide, and bor₂ represents a suitable boronic acid group or a halide, e.g. bromide or iodide for coupling, under conditions suitable for boronic acid coupling, e.g. using palladium (0) and sodium carbonate.

According to a second general process (B), a compound of formula (I) may be prepared by reaction of a compound of formula (IV) with a compound of formula (XIII)

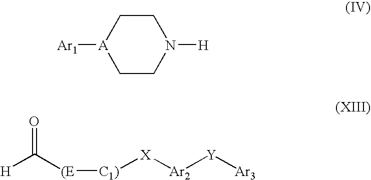

where E−C₁ (‘E minus C₁’) means that the chain length of group E is one carbon less than that in the resulting compound (I), under standard reductive amination conditions, e.g. sodium triacetoxyborohydride and acetic acid in a suitable solvent, such as dichlorom thane.

A compound of formula (XIII) may be prepared by reaction of a compound of formula (XIV) with a compound of formula (XV)

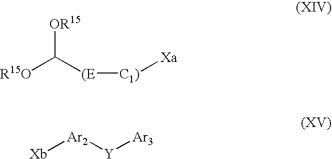

where R¹⁵ is a suitable alkyl protecting group for oxygen, such as methyl, and Xa and Xb are suitable reactants to form a group X, as defined hereinbefore, followed by removal of the protecting group, under acidic conditions.

According to a third general process (C), a compound of formula (I) may be prepared by reaction of a different compound of formula (I), by well known methods. For example a compound of formula (I) where Ar₁ is substituted by C₁₋₄alkoxy may be prepared from the corresponding compound of formula (I) where the substituent is hydroxy by standard O-alkylation methods.

Compounds of formula (V), (VI), (VII), (VII), (IX), (XI), (XIV) and (XV), are known or may be prepared by standard methods, e.g. as substantially described herein.

The protecting groups used in the preparation of compounds of formula (I) may be used in conventional manner. See for example ‘Protective Groups in Organic Chemistry’ Ed. J. F. W. McOmie (Plenum Press 1973) or ‘Protective Groups in Organic Synthesis’ by Theodora W Greene and P M G Wuts (John Wiley and Sons 1991).

Conventional amino protecting groups may include for example aralkyl groups, such as benzyl, diphenylmethyl or triphenylmethyl groups; and acyl groups such as N-benzyloxycarbonyl or t-butoxycarbonyl.

Conventional carboxylic acid protecting groups include methyl and ethyl groups.

The invention is further described with reference to the following non-limiting examples.

Abbreviations:

THF—Tetrahydrofuran, BF₃—Et₂O—Boron trifluoride diethyl etherate, DCM—Dichloromethane, TEA—triethylamine, EtOH—Ethanol, EtOAc—Ethyl acetate, IPr₂O—Di-isopropyl ether, TFA—Trifluoroacetic acid, Pd/C—Palladium on carbon, Et₂O—diethyl ether, IPrOH—Isopropanol, IprNH₂—Isopropylamine, NH₄OH—Ammoniaque, Chex—cyclohexane, MeOH—Methanol, DMF—Dimethyl formamide, EDCl—1-(3-dimethylaminopropyl)-, ethylcarbodiimide hydrochloride, HOBt—1-Hydroxybenzotriazole, MeCN—Acetonitrile, rt—Room temperature, CDl—Carbonyle diimidazole, nBuOH—nButanol, AcOH—Acetic acid CH₃SO₃H—Methane sulfonic acid, MgSO₄—Magnesium sulfate, Na₂SO₄—Sodium sulfate, HATU-O-(7-Azabenzotriazol-1-yl)-N,N,N′N′-hetramethyluroniumhexafluorophosphate

Intermediate 1

4-(4-Chloro-benzoylamino)-benzoic Acid Ethyl Ester

A solution of 4-Amino-benzoic acid ethyl ester (124.0 g, 0.75 mol) in THF/DCM (500 ml/1000 mL) was treated with TEA (120 mL, 1.15 eq.) and 4-Dimethylaminopyridine (1.3 g, catalytic amount). At −7° C. a solution of 4-Chlorobenzoyl chloride (152 g, 1.15 eq.) in THF (100 mL) was added dropwise. The resulting mixture was stirred mechanically for 48 hours. The solvent was evaporated off and the residue was taken up in EtOAc/DCM (30/70). A concentrated NaOH solution was added until pH=12. A white solid precipated out and was collected (156.8 g, 0.52 mol). The organic layer was dried over Na₂SO₄. The solvent was evaporated off and crystallization from iPr₂O gave a second batch of the title compound (63.2 g, 0.21 mol).

¹H NMR (CDCl₃, 250 MHz) δ 8.1 (s, 1H), 7.9 (d, 2H), 7.7 (d, 2H), 7.6 (d, 2H), 7.3 (d, 2H), 4.3 (q, 2H), 1.3 (t, 3H).

Intermediate 2

4-(4-Chloro-benzoylamino)-benzoic Acid

A suspension of intermediate 1 (220 g, 0.72 mol) in 2000 mL of EtOH was treated with a 1N NaOH solution (1000 mL). The resulting suspension was, heated at reflux overnight. A white solid precipated out. At reflux, concentrated HCl solution was added until pH=1. Under rigorous mechanical stirring, the resulting suspension was cooled down. A white solid was collected and dried under reduced pressure to give the title compound in a quantitative yield.

¹H NMR (DMSO, 250 MHz) δ 10.5 (s, 1H), 7.9 (d, 2H), 7.8 (s, 4H), 7.5 (d, 2H). Ref: J. Pharm. Sci. (1979), 68(3), 332-5

Intermediate 3

4-(4-chloro-benzoylamino)-N-[4-(4,4-diethoxy-butyl)]-benzamide

To a solution of intermediate 2 (10.0 g, 36.3 mmol) in DMF (60 mL), was added 4-Aminobutyraldehyde diethyl acetal (6.44 g, 1.1 eq.), HOBt (7.35 g, 1.5 eq.), CDI (8.8 g, 1.5 eq.) and TEA (7.5 mL, 1.5 eq.). The reaction was stirred at rt for 24 hours. A precipitate was formed. Water (50 mL) was added and the reaction was filtered off. The precipitate was washed with H₂O and dried to give the title compound (11.0 g, 26 mmol) as a white solid.

¹H NMR (DMSO, 250 MHz) δ 10.6 (s, 1H), 8.45 (t, 1H), 8.1 (d, 2H), 7.9 (s, 4H), 7.7 (d, 2H), 4.55 (m, 1H), 3.7-3.3 (m, 6H), 1.7 (m, 4H), 1.15 (t, 6H).

Intermediate 4

4-(4-chloro-benzoylamino)-N-[4-(4-oxo-butyl)]-benzamide

To a suspension of intermediate 3 (11.0 g, 26 mmol) in acetone (100 mL) was added a 1N HCl solution (50 mL). The reaction was stirred at reflux for 2 hours. The solvent was evaporated off and the aqueous phase was treated with a saturated NaHCO₃ solution until PH=9-11. The precipitate was filtered off, washed with H₂O and dried to give the title compound (8.3 g, 24 mmol) as a white powder.

MP: 220° C.

Intermediate 5

2-(1−Benzyl-1,2,3,6-tetrahydro-pyridin-4-yl)-5-methyl-phenol

To a solution of m-Cresol (20.0 g, 0.185 mol) and 1-Benzyl-4-piperidone (35.0 g, 1.0 eq.) was added dropwise BF₃-Et₂O (71 mL, 3.0 eq). The mixture was stirred at 100° C. for 24 hours. After cooling to rt, the mixture was treated with a 1N HCl solution (400 mL). The resulting solution was extracted with DCM. The organic layer was dried over Na₂SO₄ and evaporated to dryness to give an oil which was crytallized in cyclohexane to give the title compound (40.0 g, 0.14 mol) as a yellow powder.

GC/MS:M+ C₁₉H₂₁NO 279

Intermediate 6

5-Methyl-2-piperidin-4-yl-phenol

To a solution of intermediate 5 (40.0 g, 0.14 mol) in EtOH (600 mL) and THF (50 mL) was added Pd/C, 10% (4 g) and the reaction was stirred under an atmospheric pressure of hydrogen at 50° C. for 56 hours. The reaction mixture was filtered through a bed of celite. The filtrate was evaporated under reduced pressure to give the title compound as a white powder in a quantitative yield.

GC/MS: M+ C₁₂H₁₇NO 191

Intermediate 7

2-(1-Benzyl-1,2,3,6-tetrahydro-pyridin-4-yl)-5-ethyl-phenol

A solution of 3-Ethyl-phenol (6.1 g, 0.05 mol) and 1-Benzyl-4-piperidone (10.0 g 1.05 eq.) in acetic acid (100 mL) was treated with HCl gaz for 10 min. The mixture was stirred at 95° C. for 30 min. After cooling to rt, the mixture was treated again with HCl gaz for 5 min. The resulting solution was allowed to stir at rt for 4 days. The solvent was evaporated under reduced pressure and the residue was diluted with H₂O and extracted with DCM. The organic layer was washed with a 2N NaOH solution, H₂O and brine, dried over Na₂SO₄ and evaporated to dryness. The residue was flash chromatographed using MeOH/DCM (5/95) to give the title compound (8.0 g, 0.027 mol) as a yellow oil in 54% yield.

GC/MS: M+ C₂₀H₂₃NO 293

Intermediate 8

5-Ethyl-2-piperidin-4-yl-phenol

To a solution of intermediate 7 (8.0 g, 0.027 mol) in EtOH (100 mL) was added Pd/C, 10% (0.8 g) and the reaction was stirred under an atmospheric pressure of hydrogen for 24 hours. The reaction mixture was filtered through a bed of celite. The filtrate was evaporated under reduced pressure to give the title compound (4.9 g, 0.024 mol) as a yellow oil in a 88% yield.

GC/MS: M+ C₁₃H is NO 205

Intermediate 9

1-Benzyl-4-[2-(tert-butyl-dimethyl-silanyloxy)-4-ethyl-phenyl]-1,2,3,6-tetrahydropyridine

To a solution of intermediate 7 (3.0 g, 0.01 mol) in DMF (20 mL) was added at 50° C. NaH (1.1 eq.) (60% in oil dispersion). The reaction was stirred for 15 min and the terbutyl dimethyl silyl chloride (1.65 g, 0.011 mol) was added and the reaction was stirred for 18 hours at rt.

The reaction was concentrated in vacuo and the residue was diluted with DCM, washed with water, dried over Na₂SO₄ and evaporated off. The title compound was obtained (3.1 g, 7.6 mmol) as a yellow oil in a 77% yield.

GC/MS: M+ C₂₆H₃₇NOSi 407

Intermediate 10

4-[2-(tert-Butyl-dimethyl-silanyloxy)-4-ethyl-phenyl]-piperidine

The same method was employed as in the preparation of intermediate 8 but starting from intermediate 9 gave the title compound as an oil in a 83%.

GC/MS: M+ C₁₉H₃₃NOSi 319

Intermediate 11

2-(4-(4-[2-(tert-Butyl-dimethyl-silanyloxy)-4-ethyl-phenyl]-piperidin-1-yl)-butyl)-isoindole-1,3-dione

A solution of intermediate 10 (2.0 g, 6.2 mmol) in acetone (800 mL) was treated with K₂CO₃ (1.7 g, 2.0 eq.) and N-(4-Bromobutyl)-phtalimide (2.1 g, 1.2 eq.). The resulting mixture was stirred under reflux for 6 hours. After cooling to rt the reaction mixture was filtered off. The cake was washed with acetone. The filtrate was evaporated off to give after flash chromatography using (DCM/MeOH, 95/5) as eluent the title compound (2.1 g, 4 mmol) as yellow crystals in a 66% yield.

GC/MS: M+ C₃₁H₄₄N₂O₃Si 520

Intermediate 12

4-{4-[2-(tert-Butyl-dimethyl-silanyloxy)-4-ethyl-phenyl]-piperidin-1-yl}-butylamine

A solution of intermediate 11 (2.1 g, 4 mmol) in MeOH (50 mL) was treated with hydrazine hydrate (0.23 mL, 1.2 eq.). The resulting mixture was stirred at 60° C. for 5 hours. After evaporation under reduced pressure the residue was taken up in water and treated with a concentrated HCl solution until PH=4. The white precipitate was filtered off, washed with water and the filtrate was treated with a concentrated NaOH solution until PH=13. Extraction with DCM, drying over Na₂SO₄ and filtration gave the title compound (0.7 g, 1.8 mmol) as a yellow oil in a 45% yield.

GC/MS: M+ C₂₃H₄₂N₂OSi 390

Intermediate 13

4′-Cyano-biphenyl-4-carboxylic Acid (4-{4-[2-(tert-butyl-dimethyl-silanyloxy)-4-ethyl-phenyl-piperidin-1-yl}-butyl)-amide

To a solution of intermediate 12 (0.7 g, 1.8 mmol) in dry DCM (25 mL) was added the available 4′-Cyano-biphenyl-4-carboxylic acid (0.36 g, 0.9 eq.), EDCl (0.68 g, 2.0 eq.), HOBt (0.48 g, 2.0 eq.) and TEA (0.5 mL, 2.0 eq.). The resulting mixture was stirred for 5 hours at rt. The residue was washed with water and brine. The organic layer was dried over Na₂SO₄ and evaporated off. Purification by flash chromatography using DCM/MeOH, 90/10 as eluent gave the title compound (0.7 g, 1.17 mmol) as white crystals in a 73% yield.

MP: 140° C.

LC/MS: [M+H⁺] 596 C₃₇H₄₉N₃O₂Si

Intermediate 14

1-[4-(1-Hydroxy-5,6,7,8-tetrahydro-naphtalen-2-yl)-3,6-dihydro-2H-pyridin-1-yl]-ethanone

The same method was employed as in the preparation of intermediate 5 but starting from the 5,6,7,8-tetrahydro-1-naphtol and N-Acetyl-piperidone to give the title compound as a powder after crystallization in CH₃CN in a 100% yield.

GC/MS: M+ C₁₇H₂₁NO₂ 271

Intermediate 15

1-[4-(1-Hydroxy-5,6,7,8-tetrahydro-naphtalen-2-yl)-piperidin-1-yl]-ethanone

To a solution of intermediate 14 (55.0 g, 0.203 mol) in AcOH (500 mL) was added Pd/C, 10% (2 g) and the reaction was stirred under an atmospheric pressure of hydrogen at 50° C. for 24 hours. The mixture was filtered through a bed of celite. The filtrate was evaporated under reduced pressure to give the title compound (55.0 g, 0.201 mol) as a yellow powder.

GC/MS: M+ C₁₇H₂₂NO₂ 273

Intermediate 16

2-Piperidin-4-yl-5,6,7,8-tetrahydro-naphtalen-1-ol

To a solution of intermediate 15 (27.0 g, 0.099 mol) in EtOH (750 mL) was added a solution of NaOH (250 mL) in H₂O (250 mL). The reaction was stirred under reflux for 16 hours. After cooling, the reaction was concentrated under reduced pressure, was diluted with DCM and washed with water. The organic layer was dried over Na₂SO₄ and evaporated to dryness to give after flash chromatography using DCM/MeOH/NH4OH 30, 30, 30 as eluent, the title compound (9.7 g, 0.042 mol) as a pink gummy oil in a 42.5% yield.

¹H NMR (CDCl₃, 300 MHz) δ 7.9 (bs, 1H), 6.8 (d, 1H), 6.6 (d, 1H), 3.4 (m, 2H), 3.1 (m, 2H), 2.8 (m, 4H), 1.8-1.4 (m, 10H).

Intermediate 17

2-(4,4-Diethoxy-butyl)-isoindole-1,3-dione

To a solution of Isobenzofuran-1,3-dione (10.0 g, 0.068 mol) in toluene (200 mL) were added 4-Aminobutyraldehyde diethyl acetal (14.5 g, 1.2 eq.) and TEA (14.0 mL, 1.5 eq.). The reaction was stirred to reflux for 16 hours. The toluene was removed under vacuo and the residue was dissolved in Et₂O and washed with water. The organic phase was dried over Na₂SO₄ and concentrated under vacuo to give the title compound (21.0 g, 1.0 eq.) as an oil in a quantitative yield.

GC/MS: M+ C₁₆H₂₁NO₄ 291

Intermediate 18

4-(1,3-Dioxo-1,3-dihydro-isoindole-2-yl)-butyraldehyde

To a solution of intermediate 17 (21.0 g, 0.068 mol) in acetone (200 mL) was added a 1N HCl solution (100 mL) and the reaction was stirred to reflux for 2 hours. The solvent was then evaporated and a 1N NaOH solution (200 mL) was added. The product was extracted with DCM and the organic phase was dried over Na₂SO₄ and concentrated under vacuo. The title compound was obtain d as a yellow oil (8.4 g, 0.039 mol) in a 59% yield.

¹H NMR (CDCl₃, 300 MHz) δ 9.6 (s, 1H), 7.8 (m, 2H), 7.4 (m, 2H), 3.6 (t, 2H), 2.4 (t, 2H), 1.8 (m, 2H).

Ref: J. Med. Chem. (1992), 35, 3239-46.

Intermediate 19

2-{4-[4-(1-Hydroxy-5,6,7,8-tetrahydro-naphtalen-2-yl)-piperidin-1-yl]-butyl}-isoindole-1,3-dione

The same method was employed as in the preparation of example 1 but starting from the intermediates 16 and 18 to give after flash chromatography using (DCM/MeOH, 90/10 and 1% ammoniac solution) as eluent, the title compound as a gummy oil in a 46% yield.

¹H NMR (CDCl₃, 300 MHz) δ 7.9 (m, 2H), 7.75 (m, 2H), 6.9 (d, 1H), 6.8 (d, 1H), 6.4 (bs, 1H), 3.85 (m, 2H), 3.5 (m, 2H), 3.0 (m, 1H), 2.9 (m, 2H), 2.8 (m, 2H), 2.5 (m, 4H), 2.1 (m, 2H), 1.87 (m, 10H).

Intermediate 20

2-[1-(4-Amino-butyl)-piperidin-4-yl]-5,6,7,8-tetrahydro-naphtalen-1-ol

The same method was employed as in the preparation of intermediate 12 but starting from intermediate 19 to give the title compound as a red oil in a 90% yield.

¹H NMR (CDCl₃ 300 MHz) δ 7.0 (d, 1H), 6.6 (d, 1H), 3.1 (m, 2H), 2.9 (m, 1H), 2.65 (m, 4H), 2.6 (m, 2H), 2.45 (m, 2H), 2.1 (m, 2H), 1.85 (m, 8H), 1.5 (m, 6H).

Intermediate 21

1-[4-(1−Hydroxy-naphtalen-2-yl)-3,6-dihydro-2H-pyridin-1-yl]ethanone

The same method was employed as in the preparation of intermediate 14 but starting from the 1-Naphtol gave the title compound as a white solid in a 54% yield.

GC/MS: M+ C₁₇H₁₇NO₂ 267

Intermediate 22

1-[4-(1-Hydroxy-naphtalen-2-yl)-piperidin-1-yl]-ethanone

A solution of intermediate 21 (29.0 g, 0.112 mol) in a mixture of cyclohexene (450 mL), MeOH (100 mL), THF (350 mL) was treated with Pd(OH)₂, 50% (14 g). The resulting solution was allowed to stir at reflux for 4 days. After cooling, the reaction mixture was filtered through a bed of celite. The filtrate was evaporated to dryness to give the title compound as a white solid (22.0 g, 0.082 mol) in a 73% yield after recrystallization from CH₃CN.

LC/MS: [M+H+] C₁₇H₁₉NO₂ 270

Intermediate 23

2-Piperidinyl-naphtalen-1-ol

The same method was employed as in the preparation of intermediate 16 but starting from the intermediate 22 gave the title compound as a brown solid in a quantitative yield.

¹H NMR (DMSO, d⁶, 300 MHz) δ 9.3 (s, 1H), 8.25 (dd, 1H), 7.8 (dd, 1H), 7.5 (m, 3H), 7.25 (m, 1H), 3.45 (m, 3H), 3.1 (m, 2H), 2.9 (m, 4H).

Intermediate 24

2-{4-[4-(1-Hydroxy-naphtalen-2-yl)-piperidin-1-yl]-butyl}isoindole-1,3-dione

The same method was employed as in the preparation of intermediate 19 but starting from the intermediate 23 gave the title compound as a pink solid in a 61% yield.

¹H NMR (CDCl₃, 300 MHz) δ 8.3 (dd, 2H), 7.95 (m, 2H), 7.8 (m, 3H), 7.6-7.2 (m, 4H), 3.85 (m, 2H), 3.25 (m, 2H), 2.85 (m, 2H), 2.55 (m, 2H), 2.35 (m, 2H), 1.95 (m, 2H), 1.8 (m, 4H).

Intermediate 25

2-[1-(4-Amino-butyl)-piperidin-4-yl]-naphtalen-1-ol

The same method was employed as in the preparation of intermediate 12 but starting from intermediate 24 to give the title compound as a yellow solid in a 79% yield.

LC/MS(ES): M+ C₁₉H₂₆N₂O 298

Intermediate 26

4′-Acetyl-biphenyl-4-carboxylic Acid Ethyl Ester

To a solution of 16 g (0.058 mol.) of 4-iodo-benzoic acid ethyl ester in toluene (200 mL) was added successively 3.35 g (0.05 q.) of tetrakis (triphenylphosphine) palladium (0), 69 ml of a 2M solution of Na₂CO₃ and 7.5 g (3 eq.) of lithium chloride. After 15 minutes of stirring was added a solution of 10 g (1.05 eq.) of 4-acetylphenyl boronic acid in EtOH (50 mL). Then, the mixture was refluxed for 24 hours. After cooling, the solvents were evaporated to dryness. The residue was poured in water (300 mL) and the organic phase was separated, dried over Na₂SO₄ and evaporated off. After purification by flash chromatography (using DCM as eluent), the tilte compound (12.0 g, 0.045 mol) was obtained as a powder in a 73% yield.

GC/MS: M+ C₁₇H₁₆O₃ 268

Intermediate 27

4′-Acetyl-biphenyl-4-carboxylic Acid

To a solution of intermediate 26 (12.0 g, 0.045 mol) in EtOH (200 mL) was added a 1N NaOH solution (85 mL, 2 eq.) and the reaction was reflux for 16 hours. After cooling, the reaction was concentrated in vacuo and a 1N HCl solution (100 mL) was added. The precipitate obtained was filtered off, washed with water and dried to give the title compound as a colorless powder (10 g, 0.042 mol) in a 93% yield

GC/MS: M+C₁₅H₁₂O₃ 240

Intermediate 28

1-Benzyl-4-[2-(tert-butyl-dimethyl-silanyloxy)-4-methyl-phenyl]-1,2,3,6-tetrahydro-pyridine

The same method was employed as in the preparation of intermediate 9 but starting from the intermediate 5 gave the title compound as a yellow oil in a 30% yield.

GC/MS: M+ 393 C₂₅H₃₅NOSi

Intermediate 29

4-[2-(tert-Butyl-dimethyl-silanyloxy)-4-methyl-phenyl]-pyridine

The same method was employed as in the preparation of intermediate 8 but starting from intermediate 28 gave the title compound as a white powder oil in a quantitative yield.

GC/MS: M+ C₁₈H₃₁NOSi 305

Intermediate 30

2-(4-{4-[2-(tert-Butyl-dimethyl-silanyloxy)-4-methyl-phenyl]-pyridin-1-yl}-butyl)-isoindole-1,3-dione

The same method was employed as in the preparation of intermediate 11 but starting from intermediate 29 gave the title compound as a yellow oil in a 40% yield which crystallise in MeOH.

GC/MS: M+ C₃₀H₄₂N₂O₃Si 506

Intermediate 31

2-{4-[4-(4-Methyl-2-Hydroxy-phenyl)-piperidin-1-yl]-butyl}-isoindole-1,3-dione

The same method was employed as in the preparation of example 6 but starting from intermediate 30 gave the title compound as a yellow crystals in a 97% yield.

GC/MS: M+ C₂₄H₂₈N₂O₃ 392

Intermediate 32

Phosphoric Acid 2-{1-[4-(1,3-dioxo-1,3-dihydro-isoindol-2-yl)-butyl]-piperidin-4-yl}-5-methyl-phenyl Ester Diethyl Ester

To a solution of intermediate 31 (0.75 g, 1,9 mmol) in THF (5 mL) was added the diethyl cyano phosphonate (0.43 mL, 1.5 eq.) and the triethyl amine (0.79 mL, 3 eq.). The reaction was stirred at rt for 2 hours. Then water was added and the reaction was decanted. The aqueous phase was extracted with AcOEt and the combined organic phases were dried over Na₂SO₄ and evaporated off.

The title compound (0.75 g, 1.4 mmol) was obtained as a yellow oil in a 75% yield.

GC/MS (APCI): [M+H+] C₂₈H₃₇N₂O₆P 529

Intermediate 33

Phosphoric Acid 2-[1-(4-amino-butyl)-piperidin-4-yl]-5-methyl-phenyl Ester Diethyl Ester

The same method was employed as in the preparation of intermediate 12 but starting from intermediate 32 gave the title compound as a yellow oil in a 77% yield.

LC/MS (APCI): [M+H+] C₂₀H₃₅N₂O₄P 399

Intermediate 34

4-{4-[2-(tert-Butyl-dimethyl-silanyloxy)-4-methyl-phenyl]-pyridin-1-yl}-butylamine

The same method was employed as in the preparation of intermediate 12 but starting from intermediate 30 gave the title compound as a yellow oil in a 96% yield.

LC/MS (APCI): [M+H+] C₂₂H₄₀N₂OSi 377

Intermediate 35

4′-Cyano-biphenyl-4-carboxylic Acid (4-{4-[2-(tert-butyl-dimethyl-silanyloxy)-4-methyl-phenyl-piperidin-1-yl}-butyl)-amide

The same method was employed as in the preparation of intermediate 13 but starting from intermediate 34 gave the title compound as a white oil in a 36% yield.

LC/MS (APCI): [M+H+] C₃₆H₄₇N₃O₂Si 582

Intermediate 36

2-{4-[4-(4-Ethyl-2-Hydroxy-phenyl)-piperidin-1-yl]-butyl}-isoindole-1,3-dione

The same method was employed as in the preparation of example 6 but starting from intermediate 11 gave the title compound as a yellow crystals in a quantitative yield.

LC/MS: [M+H+] C₂₅H₃₀N₂O₃ 407

Intermediate 37

Phosphoric Acid 2-{1-[4-(1,3-dioxo-1,3-dihydro-isoindol-2-yl)-butyl]-piperidin-4-yl}-5-ethyl-phenyl Ester Diethyl Ester

The same method was employed as in the preparation of intermediate 32 but starting from intermediate 36 gave the title compound as a yellow oil in a 89% yield.

LC/MS (APCI): [M+H+] C₂₉H₃₉N₂O₆P 543

Intermediate 38

Phosphoric Acid 2-[1-(4-amino-butyl)-piperidin-4-yl]-5-ethyl-phenyl Ester Diethyl Ester

The same method was employed as in the preparation of intermediate 12 but starting from intermediate 37 gave the title compound as a yellow oil in a 63% yield.

LC/MS (APCI): [M+H+] C₂₁H₃₇N₂O₄P 413

### EXAMPLE 1

4-(4-chloro-benzoylamino)-N-{4-[4-(5-methyl-2-piperidinyl-phenol)]-butyl}-benzamide Hydrochloride

To a solution of intermediate 6 (3.0 g, 15.7 mmol) in dry THF (70 mL) and MeOH (200 mL) was added the intermediate 4 (5.4 g, 1.0 eq.). The reaction was stirred at rt for 30 min and AcOH (1.5 eq) was added. Then sodium triacetoxyborohydride (1.2 eq.) was added and the reaction was stirred for 24 hours at 80° C. After cooling, the solvent was evaporated and H₂O was added. The precipitate was filtered off, treated with a 1N HCl solution and dried to give the title compound as a white powder in 76% yield.

MP: 254° C.

Analysis for C₃₀H₃₄ClN₃O₃ (1.4 HCl)

Calculated: C, 63.09; H. 6.25; N, 7.36. Found: C, 63.26; H, 6.49; N, 7.47

### EXAMPLE 2

Acetic Acid 2-(1-{4-[4-(4-chloro-benzoylamino)-benzoylamino]-butyl}-piperidin-4-yl)-5-methyl-phenyl Ester

To a solution of example 1 (0.33 g, 0.63 mmol) in DCM (10 mL) was added an excess of anhydride acetic solution (2 mL) and pyridine (2 mL). The reaction was stirred at rt for 16 hours. Then the reagents were evaporated under reduced pressure. The precipitate was washed in hot Et₂O, filtered off, and dried to give the title compound as a colorless powder in 84% yield.

MP: 210° C.

LC/MS: [M+H⁺] 562 C₃₂H₃₆ClN₃O₄

### EXAMPLE 3

Phosphoric Acid 2-(1-{4-[4-(4-chloro-benzoylamino)-benzoylamino]-butyl}-piperidin-4-yl)-5-methyl-phenyl Ester Diethyl Ester

To a solution of example 1 (0.50 g, 0.96 mmol) in pyridine (50 mL) was added an excess of diethyl chlorophosphate (2.2 mL). The reaction was stirred at rt for 3 hours. Then the reagents were evaporated under reduced pressure. The residue was dissolved in DCM, washed with water, dried over Na₂SO₄ and evaporated under reduced pressure. After purification by flash chromatography using DCM/MeOH 98/2 and 90/10 as eluent, the title compound was obtained after crystallization from isopropyl ether in a 83% yield as a white solid.

MP: 230° C.

LC/MS: [M+H⁺] 656 C₃₄H₄₃ClN₃O₆P

### EXAMPLE 4

4-(4-chloro-benzoylamino)-N-{4-[4-(5-ethyl-2-piperidin-4-yl-phenol)]-butyl}-benzamide Acetate

The same method was employed as in the preparation of example 1 but starting from intermediate 8 gave the title compound as a white solid after recrystallization from MeOH in 64% yield.

MP: 213° C.

Analysis for C₃₁H₃₆ClN₃O₃ (1 CH₃CO₂H)

Calculated: C, 69.78; H, 7.41; N, 7.18. Found: C, 69.91; H, 7.45; N. 7.16

### EXAMPLE 5

Acetic Acid 2-(1-{4-[4-(4-chloro-benzoylamino)-benzoylamino]-butyl}-piperidin-4-yl)-5-ethyl-phenyl Ester

The same method was employed as in the preparation of example 2 but starting from example 4 gave the title compound as a colorless solid after crystallization from Et₂O in 75% yield.

MP: 192-194° C.

LC/MS (APCI): [M+H+] 576 C₃₃H₃₈ClN₃O₄

### EXAMPLE 6

4′-Cyano-biphenyl-4-carboxylic Acid {4-[4-(2-hydroxy-4-ethyl-phenyl)-piperidin-1-yl]-butyl}-amide

To a solution of intermediate 13 (0.1 g, 0.17 mmol) in THF (10 mL) was added the tetrabutylammonium fluoride (1.2 eq.). The reaction was stirred to rt during 15 min. Then H₂O (10 mL) was added and the organic phase was decanted, dried over Na₂SO₄ and vaporated off. The title compound was obtained as white crystals (0.055 g, 0.1 mmol) after recrystallization from MeOH in a 68% yield.

MP: 252° C.

LC/MS (APCI): [M+H⁺] 482 C₃₁H₃₅N₃O₂

### EXAMPLE 7

Acetic Acid 2-(1-{4-[(4′-cyano-biphenyl-4-carbonyl)-amino]-butyl}-piperidin-4-yl)-5-ethyl-phenyl Ester

The same method was employed as in the preparation of example 2 but starting from example 6 to give the title compound as a white powder in a 79% yield.

LC/MS(ES): M+ 523 C₃₃H₃₇N₃O₃

### EXAMPLE 8

4-(4-Chloro-benzoylamino)-N-{4-[4-(1-hydroxy-5,6,7,8-tetrahydro-naphtalen-2-yl)-piperidin-1-yl]-butyl}-benzamide Hydrochloride

The same method was employed as in the preparation of intermediate 13 but starting from intermediate 2 and 20 to give the title compound as white crystals after formation of chlorhydrate from a hot HCl 1N/EtOH solution in a 52% yield.

MP: 268° C.

LC/MS(ES): M+ 559 C₃₃H₃₈ClN₃O₃

### EXAMPLE 9

Acetic Acid 2-(1-{4-[4-(4-chloro-benzoylamino)-benzoylamino]-butyl}-piperidin-4-yl)-5,6,7,8-tetrahydro-naphtalen-1-yl Ester

The same method was employed as in the preparation of example 2 but starting from example 8 to give the title compound as a yellow powder in a 99% yield.

MP: 254° C.

LC/MS(ES): M+ 601 C₃₅H₄₀ClN₃O₄

### EXAMPLE 10

4′-Cyano-biphenyl-4-carboxylic Acid {4-[4-(1-hydroxy-5,6,7,8-tetrahydro-naphtalen-2-yl)-piperidin-1-yl]-butyl}-amide

The same method was employed as in the preparation of intermediate 13 but starting from intermediate 20 to give the title compound as a white powder after formation of chlorhydrate from a hot HCl 1N/EtOH solution in a 49% yield.

MP: 252° C.

LC/MS(ES): M+507 C₃₃H₃₇N₃O₂

### EXAMPLE 11

Acetic Acid 2-(1-{4-[(4′-cyano-biphenyl-4-carbonyl)-amino]-butyl}-piperidin-4-yl)-5,6,7,8-tetrahydro-naphtalen-1-yl Ester

The same method was employed as in the preparation of example 2 but starting from example 10 to give the title compound as a colorless solid in a 97% yield.

MP: 140-150° C.

LC/MS (APCI): [M+H⁺] 550 C₃₅H₃₉N₃O₃

### EXAMPLE 12

4-(4-Chloro-benzoylamino)-N-{4-[4-(1-hydroxy-naphtalen-2-yl)-piperidin-1-yl]-butyl}-benzamide Hydrochloride

The same method was employed as in the preparation of example 8 but starting from intermediate 25 to give the title compound as a white powder in a 58% yield.

MP: 274° C.

LC/MS (APCI): [M+H⁺] 550 C₃₃H₃₄N₃O₃Cl

### EXAMPLE 13

Acetic Acid 2-(1-{4-[4-(4-chloro-benzoylamino)-benzoylamino]-butyl}-piperidin-4-yl)-naphtalen-1-yl Ester

The same method was employed as in the preparation of example 2 but starting from example 12 to give the title compound as a colorless solid in a 94% yield.

MP: 240° C.

LC/MS (APCI): [M+H⁺] 598 C₃₅H₃₆N₃O₄Cl

### EXAMPLE 14

4′-Cyano-biphenyl-4-carboxylic Acid {4-[4-(1-hydroxy-naphtalen-2-yl)-piperidin-1-yl]-butyl}-amide

The same method was employed as in the preparation of intermediate 13 but starting from intermediate 25 to give the title compound as a colorless solid in a 55% yield.

MP: 135-140° C.

LC/MS (APCI): [M+H⁺] 504 C₃₃H₃₃N₃O₂

### EXAMPLE 15

Acetic Acid 2-(1-{4-[(4′-cyano-biphenyl-4-carbonyl)-amino]-butyl}-piperidin-4-yl)-naphtalen-1-yl Ester

The same method was employed as in the preparation of example 2 but starting from example 14 to give the title compound as a colorless solid in a 70% yield.

MP: 115-120° C.

LC/MS (APCI): [M+H⁺] 546 C₃₅H₃₅N₃O₃

### EXAMPLE 16

4′-Acetyl-biphenyl-4-carboxylic Acid {4-[4-(1-hydroxy-5,6,7,8-tetrahydro-naphtalen-2-yl)-piperidin-1-yl]-butyl}-amide

The same method was employed as in the preparation of intermediate 13 but starting from intermediate 20 and 27 to give the title compound as a colorless powder after purification by flash chromatography (using DCM/MeOH 80/20 as eluent) and crystallisation in iPr₂O in a 49% yield.

MP: 180-185° C.

LC/MS (APCI): [M+H+] 525 C₃₄H₄₀N₂O₃

### EXAMPLE 17

Phosphoric Acid 2-(1-{4-[(4′-cyano-biphenyl carbonyl)-amino]-butyl}-piperidin-4-yl)-5-methyl-phenyl Ester Diethyl Ester

The same method was employed as in the preparation of intermediate 13 but starting from intermediate 33 to give the title compound as a white crystals in a 22% yield after purification by flash chromatography using DCM/MeOH 90/10 as eluent.

LC/MS (APCI): [M+H+] 604 C₃₄H₄₂N₃O₅P

### EXAMPLE 18

Phosphoric Acid mono-[2-(1-{4-[(4′-cyano-biphenyl-4-carbonyl)-amino]-butyl}-piperidin-4-yl)-5-methyl-phenyl]ester

To a solution of example 17 (0.07 g, 0.1 mmol) in DCM (2 mL), was added the bromo trimethyl silan (0.06 mL, 4 eq.) and pyridine (10 eq.) at 0° C. The reaction was stirred at rt then was heated at 60° C. for 1 hour. After cooling, water was added (2 mL) and the compound was extracted with DCM, dried over Na₂SO₄ and evaporated off. The title compound (0.02 mg, 0.036 mmol) was crystallised as yellow crystals from Et₂O in a 37% yield.

MP: 70° C.

LC/MS (APCI): [M+H+] 548 C₃₀H₃₄N₃O₅P

### EXAMPLE 19

Phosphoric Acid 2-(1-{4-[(4′-cyano-biphenyl-4-carbonyl)-amino]-butyl}-piperidin-4-yl)-5,6,7,8-tetrahydro-naphtalen-1-yl Ester Diethyl Ester

To a solution of example 10 (0.1 g, 0,18 mmol) in DMF (10 mL, was added NaH in 60% oil dispersion

(0.080 g, 1.1 eq.). The reaction was stirred at rt for 10 min and the chloro diethyl phosphonate (0.1 mL, 4 eq.) was added. The reaction was stirred at rt 16 hours and at 60° C. for 4 hours. The solvent was evaporated off and the residue was purified by flash chromatography using DCM/MeOH 98/2 and 90/10 to give the title compound (0.065 mg, 0.1 mmol) as a colorless solid which was crystallised from iPrO₂ in a 57% yield.

MP: 90° C.

LC/MS (APCI): [M+H+] 645 C₃₇H₄₆N₃O₅P

### EXAMPLE 20

4′-Cyano-biphenyl-4-carboxylic Acid {4-[4-(2-hydroxy-4-methyl-phenyl)-piperidin-1-yl]-butyl}-amide

The same method was employed as in the preparation of example 6 but starting from intermediate 35 to give the title compound as a white crystals in a 34% yield.

MP: 184° C.

LC/MS (APCI): [M+H⁺] 468 C₃₀H₃₃N₃O₂

### EXAMPLE 21

Acetic Acid 2-(1-{4-[(4′-cyano-biphenyl-4-carbonyl)-amino]-butyl}-piperidin-4-yl)-5-methyl-phenyl Ester

The same method was employed as in the preparation of example 2 but starting from example 20 to give the title compound as a yellow powder in a 79% yield.

MP: 128-130° C.

LC/MS (APCI): [M+H+] 510 C₃₂H₃₅N₃O₃

### EXAMPLE 22

Phosphoric Acid 2-(1-{4-[4-(4-chloro-benzoylamino)-benzoylamino]-butyl}-piperidinyl)-5,6,7,8-tetrahydro-naphtalen-1-yl Ester Diethyl Ester

The same method was employed as in the preparation of example 19 but starting from example 8 to give the title compound as a yellow powder in a 36% yield.

MP: 200° C.

LC/MS (APCI): [M+H+] 696 C₃₇H₄₇N₃O₆ClP

### EXAMPLE 23

Phosphoric Acid 2-(1-{4-[4-(4-chloro-benzoylamino)-benzoylamino]-butyl}-piperidin-4-yl)-5-ethyl-phenyl Ester Diethyl Ester

The same method was employed as in the preparation of intermediate 13 but starting from intermediate 2 and 38 to give the title compound as white crystals in a 38% yield.

MP: 158-160° C.

LC/MS (APCI): [M+H+] 671 C₃₅H₄₅ClN₃O₆P

Biological Assays

In Vitro Assay:

HepG₂ cells, stably transfected with a construct comprising the the LDL-r promoter and the luciferase reporter gene, were seeded at 50,000 cells/well in 96 well plates. After 1 day, cells were incubated with compounds for 24 hours in RPMI medium containing 2% of lipoprotein-deficient serum. Compounds were tested from 10⁻⁶M to 10⁻⁹M. Cell lysates were prepared and the luciferase activity was measured by the luciferase assay system (Promega). Induction of luciferase activity was calculated taking untreated cells as control and ED₅₀ of each compounds was determinated compared to the ED₅₀ of an internal standart.

In Vivo Assay:

Compounds were prepared for oral administration by milling with 0.5% hydroxypropylmethylcellulose and 5% Tween 80. Hamsters were fed for 2 weeks with a diet containing 0.2% of cholesterol and 10% of coconut oil. Then compounds were administrated once a day for 3 days, from 20 to 0.2 mg/kg. Plasma lipid levels including total cholesterol, VLDL/LDL cholesterol, VLDL/LDL triglycerides and HDL-cholesterol were determinated after ultracentrifugation (density 1.063 g/ml to separate VLDL/LDL fraction and HDL fraction) using the Biomerieux erizymatic kit. Reductions in VLDL/LDL cholesterol and TG plasmatic levels were calculated taking solvant treated animals as control and ED₅₀ of each compound was determined.

Biological Results

| Example | In vitro (IC₅₀) (nm) | In vivo (ED₅₀) (mg/kg) |
|---|---|---|
| 1 | 30 | 10-15 |
| 2 | 30 | 5 |
| 3 | 30 | 5 |
| 22 | 4 | 2-5 |

Tablet Compositions

The following compositions A and B can be prepared by wet granulation of ingredients (a) to (c) and (a) to (d) with a solution of povidone, followed by addition of the magnesium stearate and compression.

| | | mg/tablet | mg/tablet |
|---|---|---|---|
| Composition A | | | |
| (a) | Active ingredient | 250 | 250 |
| (b) | Lactose B.P. | 210 | 26 |
| (c) | Sodium Starch Glycollate | 20 | 12 |
| (d) | Povidone B.P. | 15 | 9 |
| (e) | Magnesium Stearate | 5 | 3 |
| | | 500 | 300 |
| Composition B | | | |
| (a) | Active ingredient | 250 | 250 |
| (b) | Lactose 150 | 150 | — |
| (c) | Avicel PH 101 | 60 | 26 |
| (d) | Sodium Starch Glycollate | 20 | 12 |
| (e) | Povidone B.P. | 15 | 9 |
| (f) | Magnesium Stearate | 5 | 3 |
| | | 500 | 300 |
| Composition C | | | |
| | Active ingredient | 100 | |
| | Lactose | 200 | |
| | Starch | 50 | |
| | Povidone | 5 | |
| | Magnesium Stearate | 4 | |
| | | 359 | |

The following compositions D and E can be prepared by direct compression of the admixed ingredients. The lactose used in composition E is of the direct compression type.

| | | mg/tablet |
|---|---|---|
| Composition D | | |
| | Active ingredient | 250 |
| | Magnesium Stearate | 4 |
| | Pregelatinised Starch NF15 | 146 |
| | | 400 |
| Composition E | | |
| | Active ingredient | 250 |
| | Magnesium Stearat | 5 |
| | Lactose | 145 |
| | Avicel | 100 |
| | | 500 |
| Composition F (Controlled release composition) | | |
| (a) | Active ingredient | 500 |
| (b) | Hydroxypropylmethylcellulose | 112 |
| | (Methocel K4M Premium) | |
| (c) | Lactose B.P. | 53 |
| (d) | Povidone B.P.C. | 28 |
| (e) | Magnesium Stearate | 7 |
| | | 700 |

The composition can be prepared by wet granulation of ingredients (a) to (c) with a solution of povidone, followed by addition of the magnesium stearate and compression.

Composition G (Enteric-Coated Tablet)

Enteric-coated tablets of Composition C can be prepared by coating the tablets with 25 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

Composition H (Enteric-Coated Controlled Release Tablet)

Enteric-coated tablets of Composition F can be prepared by coating the tablets with 50 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

(ii) Capsule Compositions

Composition A

Capsules can be prepared by admixing the ingredients of Composition D above and filling two-part hard gelatin capsules with the resulting mixture. Composition B (infra) may be prepared in a similar manner.

| | | mg/capsule |
|---|---|---|
| Composition B | | |
| (a) | Active ingredient | 250 |
| (b) | Lactose B.P. | 143 |
| (c) | Sodium Starch Glycollate | 25 |
| (d) | Magnesium Stearate | 2 |
| | | 420 |
| Composition C | | |
| (a) | Active ingredient | 250 |
| (b) | Macrogol 4000 BP | 350 |
| | | 600 |

Capsules can be prepared by melting the Macrogol 4000 BP, dispersing the active ingredient in the melt and filling two-part hard gelatin capsules therewith.

| | mg/capsule |
|---|---|
| Composition D | |
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

Composition E (Controlled Release Capsule)

| | mg/capsule |
|---|---|
| (a) Active ingredient | 250 |
| (b) Microcrystalline Cellulose | 125 |
| (c) Lactose BP | 125 |
| (d) Ethyl Cellulose | 13 |
| | 513 |

The controlled release capsule composition can be prepared by extruding mixed ingredients (a) to (c) using an extruder, then spheronising and drying the extrudate. The dried pellets are coated with a release controlling membrane (d) and filled into two-part, hard gelatin capsules.

| | mg/capsule |
|---|---|
| Composition F (Enteric capsule) | |
| (a) Active ingredient | 250 |
| (b) Microcrystalline Cellulose | 125 |
| (c) Lactose BP | 125 |
| (d) Cellulose Acetate Phthalate | 50 |
| (e) Diethyl Phthalate | 5 |
| | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into two-part, hard gelatin capsules.

Composition G (Enteric-Coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

(iii) Intravenous injection composition

| | |
|---|---|
| Active ingredient | 0.200 g |
| Sterile, pyrogen-free phosphate buffer (pH 9.0) to 10 ml | |

The active ingredient is dissolved in most of the phosphate buffer at 35-40° C., then made up to volume and filtered through a sterile micropore filter into sterile 10 ml glass vials (Type 1) which are sealed with sterile closures and overseals.

(iv) Intramuscular injection composition

| | | |
|---|---|---|
| Active ingredient | 0.20 | g |
| Benzyl Alcohol | 0.10 | g |
| Glycofurol 75 | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

(v) Syrup composition

| | | |
|---|---|---|
| Active ingredient | 0.25 | g |
| Sorbitol Solution | 1.50 | g |
| Glycerol | 1.00 | g |
| Sodium Benzoate | 0.005 | g |
| Flavour | 0.0125 | ml |
| Purified Water q.s. to | 5.0 | ml |

The sodium benzoate is dissolved in a portion of the purified water and the sorbitol solution added. The active ingredient is added and dissolved. The resulting solution is mixed with the glycerol and then made up to the required volume with the purified water.

(vi) Suppository composition

| | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 200 lm sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 250 lm stainless steel screen and, with continuous stirring, allowed to cool to 40° C. At a temperature of 38-40° C., 2.02 g aliquots of the mixture are filled into suitable plastic moulds and the suppositories allowed to cool to room temperature.

(vii) Pessary composition

| | mg/pessary |
|---|---|
| Active ingredient (63 lm) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

(viii) Transdermal composition

| | |
|---|---|
| Active ingredient | 200 mg |
| Alcohol USP | 0.1 ml |
| Hydroxyethyl cellulose | |

The active ingredient and alcohol USP are gelled with hydroxyethyl cellulose and packed in a transdermal device with a surface area of 10 cm².

1. A compound of formula (I) 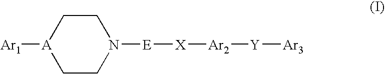
wherein
Ar₁ represents phenyl, naphthyl or phenyl fused by a C₃₋₈cycloalkyl, where each group is substituted by a group —O-Z and optionally one to three further groups independently represented by R¹;
Ar₂ represents phenyl or 5-6 membered heteroaromatic group, where each group is optionally substituted by one to four groups independently selected from halogen, C₁₋₄ alkyl and C₁₋₄ alkoxy;
Ar₃ represents a phenyl or a 5-6 membered heteroaromatic group, where each group is optionally substituted by one to four groups independently selected from hydroxy, alkyl, C₁₋₄ alkoxy, C₂₋₄ alkenyl, C₂₋₄ alkenyloxy, C₁₋₄ perfluoroalkoxy, C₁₋₄ acylamino or an electron withdrawing group;
A represents —C(H)—;
E represents —C₁₋₆ alkylene-;
X represents —CON(H or C₁₋₄alkyl)- or —N(H or C₁₋₄alkyl)CO—;
Y represents a direct link, —N(H or C₁₋₄alkyl)CO— or —CON(H or C₁₋₄alkyl)-;
Z represents a metabolically labile group;
R¹ represents halogen, —S(C₁₋₄alkyl), —O—(C₀₋₄ alkylene)-R² or —(C₀₋₄alkylene)-R², where each alkyl one group may additionally incorporate an oxygen in the chain, with the proviso that there are at least two carbon atoms between any chain heteroatoms;
R² represents
(i) hydrogen, C₁₋₄ perfluoroalkyl, C₂₋₃alkenyl,
(ii) phenyl, naphthyl, a 5- or 6-membered heteroaromatic group or 1,2,3,4-tetrahydronaphthyl, optionally substituted by one or two halogen, hydroxy, C₁₋₄ alkyl, C₁₋₄ alkoxy groups,
(iii) C₃₋₈cycloalkyl, a 3-7 membered heterocycloalkyl,
(iv) amino, C₁₋₄ alkylamino or di-C₁₋₄alkylamino, with the proviso that there are at least two carbon atoms between any chain heteroatoms;
or a physiologically acceptable salt or solvate thereof.

2. A compound according to claim 1 where Ar₁ represents a phenyl, naphthyl or 1,2,3,4-tetrahydronaphthyl group, substituted by a group —O-Z, where further optional substititution is effected by R¹.

3. A compound according to claim 1 or 2 where further substitution on Ar₁ is represented by one or two groups independently selected from C₁₋₄ alkyl, C₁₋₄ alkoxy, C₂₋₃alkenyloxy and —O-Cₒ₋₄alkylene-R², where R² represents a C₃₋₈cycloalkyl.

4. A compound according to any one of claims 1 to 3 where Z is C₁₋₄ acyl, C₁₋₄ acyloxymethylene, optionally substituted benzoyl, where optional substitution may be effected by one or more C₁₋₄ alkyl, halogen, hydroxy or C₁₋₄ alkoxy, —PO(OR³)₂, where R³ represents hydrogen, C₁₋₄ alkyl, phenyl or phenylmethyl, carboxyethylcarbonyl, C₁₋₄ alkylaminocarbonyl, C₁₋₄ dialkylaminocarbonyl or esters formed with readily available amino acids, such as dimethylaminomethylcarbonyl.

5. A compound according to claim 4 where Z is C₁₋₄ acyl, or —PO(OR³)₂, where R³ represents hydrogen, C₁₋₄ alkyl, phenyl or phenylmethyl.

6. A compound according to any one of claims 1-5 where Ar₂ is a para substituted phenyl.

7. A compound according to any one of claims 1-5 where Ar₃ is phenyl substituted by a halogen, C₁₋₄perfluoroalkyl, nitrile or C₁₋₄alkylsulfonyl.

8. A compound of formula (Ia) 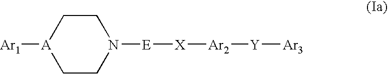
wherein
Ar₁ represents phenyl, naphthyl or phenyl fused by a C₃₋₈cycloalkyl, where each group is substituted by a group —O-Z and optionally one to three further groups independently represented by R¹;
Ar₂ represents a phenyl or 5-6 membered heteroaromatic group, where each group is optionally substituted by one to four groups independently selected from halogen, C₁₋₄ alkyl and C₁₋₄ alkoxy;
Ar₃ represents a phenyl or a 5-6 membered heteroaromatic group, where each group is optionally substituted by one to four groups independently selected from halogen, hydroxy, nitrile, C₁₋₄ alkyl, C₁₋₄ alkoxy, C₂₋₄ alkenyl, C₂₋₄ alkenyloxy, C₁₋₄ perfluoroalkyl, C₁₋₄ perfluoralkoxy, C₁₋₄ acyl, C₁₋₄ alkoxycarbonyl, aminocarbonyl, C₁₋₄ alkylaminocarbonyl; di-C₁₋₄ alkylaminocarbonyl and C₁₋₄ acylamino;
A represents —C(H)—;
E represents —C₁₋₆ alkylene-;
X represents —CON(H or C₁₋₄alkyl)- or —N(H or C₁₋₄alkyl)CO—;
Y represents a direct link, —N(H or C₁₋₄alkyl)CO— or —CON(H or C₁₋₄alkyl)-;
Z represents a metabolically labile group;
R¹ represents halogen, —O—(C₀₋₄ alkylene)-R² or —(C₀₋₄alkylene)-R², where each alkylene group may additionally incorporate an oxygen in the chain, with the proviso that there are at least two carbon atoms between any chain heteroatoms;
R² represents
(i) hydrogen, C₁₋₄ perfluoroalkyl,
(ii) phenyl, naphthyl, a 5- or 6-membered heteroaromatic group or 1,2,3,4-tetrahydronaphthyl, optionally substituted by one or two halogen, hydroxy, C₁₋₄ alkyl, C₁₋₄ alkoxy groups,
(iii) C₃₋₈cycloalkyl, a 3-7 membered heterocycloalkyl,
(iv) amino, C₁₋₄ alkylamino or di-C₁₋₄alkylamino, with the proviso there are at least two carbon atoms between any chain heteroatoms;
or a physiologically acceptable salt or solvate thereof.

10. A compound of formula (Ib) 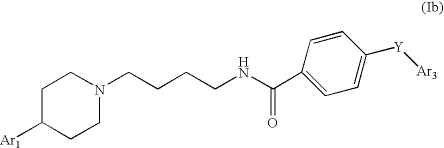
wherein
Ar₁ represents phenyl, naphthyl or 1,2,3,4-tetrahydronaphthyl, where each group is substituted in by a group —O-Z and optionally by one or two further groups independently represented by R¹;
Ar₃ represents phenyl substituted in the para position by a halogen, nitrile or C₁₋₄ perfluoroalkyl group;
R¹ represents halogen, C₁₋₄ alkyl, C₁₋₄ alkyl or O—(C₀₋₄alkylene)-R²,
R² represents hydrogen, C₁₋₄ perfluoroalkyl, a 5- or 6-membered heteroaromatic group or C₃₋₈cycloalkyl;
Y represents a direct link or —N(H)CO—;
Z represents a C₁₋₄acyl group or a PO(OR³)₂, where R³ represents hydrogen, C₁₋₄ alkyl, phenyl or phenylmethyl;
or a physiologically acceptable salt or solvate thereof.

11. A compound selected from:
Acetic acid 2-(1-{4-[4-(4-chloro-benzoylamino)-benzoylamino]-butyl}-piperidin-4-yl)-5-methyl-phenyl ester;
Phosphoric acid 2-(1-{4-[4-(4-chloro-benzoylamino)-benzoylamino]-butyl}-piperidin-4-yl)-5-methyl-phenyl ester diethyl ester;
Acetic acid 2-(1-{4-[4-(4-chloro-benzoylamino)-benzoylamino]-butyl}-piperidin-4-yl)-5-ethyl-phenyl ester;
Acetic acid 2-(1-{4-[(4′-cyano-biphenyl-4-carbonyl)-amino]-butyl}-piperidin-4-yl)-5-ethyl-phenyl ester;
Acetic acid 2-(1-{4-[4-(4-chloro-benzoylamino)-benzoylamino]-butyl}-piperidin-4-yl)-5,6,7,8-tetrahydro-naphtalen-1-yl ester;
Acetic acid 2-(1-{4-[(4′-cyano-biphenyl-4-carbonyl)-amino]-butyl}-piperidin-4-yl)-5,6,7,8-tetrahydro-naphtalen-1-yl ester;
Acetic acid 2-(1-{4-[4-(4-chloro-benzoylamino)-benzoylamino]-butyl}-piperidin-4-yl)-naphtalen-1-yl ester;
Acetic acid 2-(1-{4-[(4′-cyano-biphenyl-4-carbonyl)-amino]-butyl}-piperidin-4-yl)-naphtalen-1-yl ester;
Phosphoric acid 2-(1-{4-[(4′-cyano-biphenyl-4-carbonyl)-amino]-butyl}-piperidin-4-yl)-5-methyl-phenyl ester diethyl ester;
Phosphoric acid mono-[2-(1-{4-[(4′-cyano-biphenyl-4-carbonyl)-amino]-butyl}piperidin-4-yl)-5-methyl-phenyl]ester;
Phosphoric acid 2-(1-{4-[(4′-cyano-biphenyl-4-carbonyl)-amino]-butyl}-piperidin-4-yl)-5,6,7,8-tetrahydro-naphtalen-1-yl ester diethyl ester;
Acetic acid 2-(1-{4-[(4′-cyano-biphenyl-4-carbonyl)-amino]-butyl}-piperidin-4-yl)-5-methyl-phenyl ester;
Phosphoric acid 2-(1-{4-[4-(4-chloro-benzoylamino)-benzoylamino]butyl}-piperidinyl)-5,6,7,8-tetrahydro-naphtalen-1-yl ester diethyl ester;
Phosphoric acid 2-(1-{4-[4-(4-chloro-benzoylamino)-benzoylamino]-butyl}-piperidin-4-yl)-5-ethyl-phenyl ester diethyl ester;
or a physiologically acceptable salt or solvate thereof.

12. Use of a compound according to any one of claims 1-11 in human medicine.

13. Use of a compound according to any one of claims 1-11 or a physiologically acceptable salt or solvate thereof in the preparation of a medicament for use in the treatment of conditions resulting from elevated circulating levels of LDL-cholesterol.

14. A method for the treatment of a mammal, including man, of conditions resulting from elevated circulating levels of LDL-cholesterol, comprising administration of an effective amount of a compound according to any one of claims 1-11 or a physiologically acceptable salt or solvate thereof.

15. A pharmaceutical composition which comprises at least one compound according to any one of claims 1-11 or a physiologically acceptable salt or solvate thereof, with one or more pharmaceutically acceptable carriers or excipients and optionally one or more further physiologically active agents.

16. A process for the preparation of compound of formula (I) comprising:
(A)—reaction of a compound of formula (II) with a compound of formula (III) 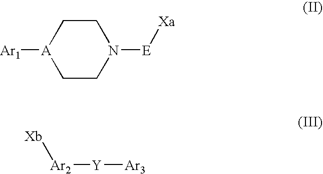
where Xa and Xb are suitable reactants to form a group X;
(B) reaction of a compound of formula (IV) with a compound of formula (XIII) 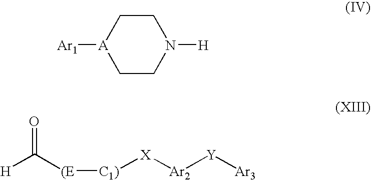
where E−C₁ (‘E minus C₁’) means that the chain length of group E is one carbon less than that in the resulting compound (I), under standard reductive amination conditions; or
(C) reaction of a different compound of formula (I).
